(19) United States
(12) Patent Application Publication
(10) Pub. No.: US 2017/0151204 A1
(43) Pub. Date: Jun. 1, 2017

(54) USE OF LEUKOTRIENE B4 IN COMBINATION WITH A TOLL-LIKE RECEPTOR LIGAND, A RIG-I-LIKE RECEPTOR LIGAND, OR A NOD-LIKE RECEPTOR LIGAND TO ENHANCE THE INNATE IMMUNE RESPONSE

(71) Applicant: Universite de Laval, Quebec (CA)

(72) Inventors: Jean Gosselin, Quebec (CA); Eric Gaudreault, Quebec (CA)

(21) Appl. No.: 15/429,872

(22) Filed: Feb. 10, 2017

Related U.S. Application Data
(63) Continuation of application No. 13/807,020, filed on May 31, 2013.

(51) Int. Cl. A61K 31/202; A61K 45/06

(52) U.S. Cl. CPC A61K 31/202; A61K 45/06

(57) ABSTRACT

The present invention relates to the use of leukotriene B₄ to enhance the response of Toll-like receptor (TLR), RIG-I-like receptor (RLR), and NOD-like receptor (NLR) when stimulated simultaneously with respective proper ligands. The use in combination of LTB₄ with those ligands is useful to potentiate immune response for the treatment of autoimmune diseases, immunosuppressive diseases, as well as immunological disorders.

This application is a continuation application of pending application U.S. Ser. No. 13/807,020 filed May 31, 2013, the entire content of which is hereby incorporated by reference.

### TECHNICAL FIELD

The present invention relates to a new combination therapy using LTB4 with at least one of a Toll-like receptor (TLR) ligand, a RIG-I-like receptor (RLR) ligand or a NOD-like receptor (NLR) ligand and a new composition for enhancing an immune response in immunosuppressed patients or in patients suffering from immunological disorders.

### BACKGROUND OF THE INVENTION

The first mention of Toll-like receptor was reported in 1997 by the Janeway group from Yale University (Medzhitov R. et al. 1997. Nature 388: 394-397). A human homologue of the fruit fly Drosophila melanogaster Toll protein was cloned and was shown to be implicated in innate immunity. Like the Drosophila Toll protein, this human Toll-like receptor (TLR) protein was a transmembrane protein with an extracellular domain containing leucine-rich repeat domains and a cytoplasmic domain structurally similar to the IL-1 receptor. The Drosophila Toll protein was shown to be implicated in immunity in the adult fly, especially in antibacterial and antifungal immunity. Similarly, the human Toll protein seemed to be implicated in cytokine secretion as well as in the expression of co-stimulatory molecules, a hallmark of naive T cell activation. In 1998 (Chaudhary P M. 1998. Blood 91: 4020-4027), both TLR3 and TLR4 were cloned and were found to be homologs of Drosophila Toll gene. These receptors, as the Janeway group demonstrated, were transmembrane receptors able to activate NFκB as well as AP-1 signalling and were found to be abundantly expressed by peripheral blood leukocytes. The first TLR ligands reported were lipopolysaccharides (LPS) from gram-negative bacteria and were associated with TLR4 signalling. From then on, a worldwide concerted effort was put forward to find more TLR candidates as well as their respective ligands. At the moment, 10 different human members of the TLR family have been identified and named TLR1 to TLR10. More than their implication with innate and adaptive immunity, TLRs are now being seen as pattern-recognition receptors implicated in the control of many human and animal pathologies. TLRs can be divided into two categories, namely cell-surface TLRs and intracellular/endosomal TLRs. TLR 1,2,4,5,6, and 10 are cell-surface receptors able to be activated by exogenous ligands, while TLR 3,7,8, and 9 are expressed intracellularly and are associated with endosomes. These receptors are therefore able to recognize endogenous ligands as well as foreign nucleic acids present inside the cell.

TLR engagement leads to different intracellular signalling pathways that have been well-characterized (Akira S. 2004. Nat. Rev. Immunol. 4: 499-511). Following ligand binding to most TLRs, except TLR3 and TLR4 in particular situations, the recruitment of the adaptor protein MyD88 to the intracellular TIR domain of the receptor is induced. In contrast, TLR3 stimulation leads to the recruitment of the adaptor protein TRIF. Other adaptor proteins are known to be implicated in TLR signalling including Mal, TIRAP, TRAM, and SRAM and are solicited in very specific occasions. Recruitment of MyD88 to the TLR intracellular domain leads to the activation of the serine-threonine kinases IL-IR associated kinase (IRAK1) and IRAK-2, which co-associate with TNFR-activated factor-6 (TRAF-6) and transforming growth factor-beta-activated kinase-1 (TAK-1). TAK-1 activation is central to TLR signalling since this kinase has the ability to activate different kinases of the mitogen-activated protein (MAP) kinase family such as p38 and c-Jun N-terminal kinases (JNK kinases) leading to AP-1-mediated gene transcription.

Moreover, TAK-1 can activate NF-κ B-inducing kinase (NIK) which in turn can lead to the recruitment of the IkB kinase complex comprised of IKKα/β/γ subunits. The formation of the IKK complex can then phosphorylate the NF-κ B inhibitory protein I-κ B and target it for degradation. Following I-κB degradation, the transcription factor NF-κB is then free to translocate to the nucleus and activate gene transcription. Signalling via the TRIF protein differs from MyD88-dependent signalling by the fact that following association of TRIF to the intracellular domain of either TLR3 or TLR4, the formation and activation of a complex comprising of TANK binding kinase 1 (TBK1), IKK_{ε}, the adaptors TANK (TNF pathway with ankyrin repeats) and TRAF3 is induced. This complex activation can then phosphorylate the interferon-responsive factors IRF3 and IRF7, which causes them to form dimers and migrate to the nucleus in order to activate interferon gene promoters.

TLR2 was first wrongly associated with LPS-mediated signalling. A careful analysis of LPS preparations presented evidence that the actual TLR2 ligands were in fact contaminating lipoproteins present in the preparation. Now it is well-established that different ligands may bind and activate TLR2. In fact, TLR2 is considered the member of the TLR family that recognizes the broadest range of structures (Zähringer U. 2008. Immunobiology 213: 205-224). The most reported ligands of such receptor are the pathogen-associated molecular patterns (PAMPs) of different microorganisms such as lipoteichoic acids (LTA), lipopeptides from natural as well as synthetic origins, lipoarabinomannans, lipomannans, glycosylphosphatidylinositol, various proteins including lipoproteins and glycoproteins, zymosan, and peptidoglycan (PGN). Following ligand binding, TLR2 is known to signal by itself or in combination with TLR1 or TLR6 co-receptors depending on the structural nature of the ligand. Ligands such as triacyl lipopeptides are prototypical ligands for TLR1/2 complex, while diacyl lipopeptides preferentially activate TLR2/6 complexes. Lipoproteins and peptidoglycans on the other hand can activate TLR2 by itself.

TLR4 is the well-known receptor binding LPS, the main structural constituent of gram-negative bacteria. LPS stimulation of TLR4 leads to an important inflammatory response from the host. In order to fully activate TLR4, LPS must first bind to the LPS-binding protein and then be recognized by the accessory molecule MD-2, which facilitates the transport of TLR4 to the cell surface. The activation of TLR4 by the S-form of LPS needs the cooperation of the cell-surface CD14 molecule for a better ligand binding and recognition. On the other hand, the LPS R-form only requires the trigger of the TLR4/MD-2 complex for full activation. Lipid A is also believed to be a natural ligand for TLR4. The production of pro-inflammatory cytokines such as TNF-α and IL-β following TLR4 activation by LPS requires the signalling induced from the recruitment of both MyD88 and TRIF, in association with Mal and TRAM proteins respectively. However, TLR4-mediated induction of type 1 interferon secretion is independent of the MyD88/Mal pathway and relies exclusively on the TRIF/TRAM signalling platform.

The flagellin protein from different bacterial species has now been reported to be a natural ligand for TLR5. Following ligand binding, TLR5 is known to dimerize and signal via MyD88-dependent, but TIRAP-independent mechanisms. Following TLR5-mediated MyD88 association, a typical intracellular activation dependent on MyD88 signalling as discussed previously is therefore activated. This cellular activation seems essential in the clearance of bacterial infection. TLR5 also seems important in the protection of the gut from enteric bacteria as well as the potential harmful actions of commensal bacteria. Loss of TLR5 can also be linked to the development of spontaneous colitis probably by the impossibility for the host to manage the commensal microflora.

Intracellular TLRs comprise TLR3, 7, 8, and 9 and signal via endosomal compartments following nucleic acid sequence binding, either being RNA or DNA. TLR7, 8, and 9 are known to signal via MyD88 adaptor protein and activate MAP kinases as well as NFκB signalling. On the other hand, TLR3 preferentially signals via TRIF recruitment leading to IRF3 and IRF7 activation for IFN gene activation.

Intracellular receptors of the RIG-I-like (RLR) and NOD-like receptor (NLR) families have recently been characterized. RIG-I-like receptor family relies on the retinoic acid-inducible gene-I (RIG-I) prototype. RIG-I structure is comprised of a helicase enzymatic activity as well as a protein-protein interaction or effector domain called caspase recruitment domain (CARD). RIG-I and alike are intracellular receptors known to bind to nucleic acid structures such as dsRNA, with RIG-I also detecting uncapped 5′-triphosphate dsRNA or ssRNA. Following RIG-1 interaction with RNA structures, facilitated by the adaptor protein IPS-1/MAVS/VISA/Cardiff, TRAF3 protein recruitment is induced leading to TBK1 activation and subsequent activation and migration of IRF3 to the nucleus for IFN gene induction. RLR signalling is important in innate immune defence mechanisms, but the involvement of these receptors in the control of other potential pathologies can be expected. Nod-like receptors (NLR) are intracellular receptors which contain a ligand-binding C-terminal leucine-rich repeat sequence, a central nucleotide-binding site, which is thought to regulate self-oligomerization, and an N-terminal protein-protein interaction or effector domain, composed of a CARD or a pyrin domain with NOD1 and NOD2 being prototypic members. NOD1/2 recognize different foreign structures such as flagellin, muramyl dipeptide and meso-diaminopimelic acid. Signalling via NLR leads to NFκB activation via the recruitment of RIP (receptor-interacting protein) 2/RICK protein or CARDIAK [CARD-containing ICE (IL-1b-converting enzyme)-associated kinase] through a CARD-CARD interaction, and this complex in turn activates the IKK complex leading to full NFκB activation. Other NLR members such as IPAF and Nalp3 are known to be components of the newly described inflammasome. An inflammasome is a multiprotein structure known to recruit and activate caspase 1, an important protein involved in the inflammatory processes such as the conversion of pro-IL-1β and pro-IL-18 to their mature form. NLR are known to be involved in inflammatory as well as defence processes. Moreover, mutations in NOD1 and NOD2 have been associated to human inflammatory disorders, including Crohn's disease, Blau syndrome, early-onset sarcoidosis, and atopic diseases.

Leukotriene B₄ (LTB₄) is a known endogenous natural molecule produced by different cells via the metabolism of arachidonic acid present in cell membranes, particularly the bilipid nuclear membrane. Arachidonic acid is converted to LTB₄ by the enzymatic activity of the 5-lipoxygenase as well as leukotriene A₄ hydrolase. This lipid has numerous well-known and characterized biological properties, especially for human leukocytes. For example, LTB₄ is an excellent chemoattractant and chemokinetic factor for human neutrophils, monocytes, macrophages, T cells, and eosinophils. LTB₄ can also lead to cell degranulation, production and secretion of reactive oxygen species, inhibition of apoptosis as well as enhanced phagocytosis. Different leukocytic cell population do possess the capacity to synthesize LTB₄ and include, but is not restricted to, neutrophils, monocytes, macrophages, and B cells. LTB₄ can bind to its two cell surface receptors of the G-protein-coupled receptor family, namely BLT1 and BLT2. Following LTB₄ ligation to BLT1/2, recruitment to the receptor of different intracellular g-proteins, particularly the G_{αi/16} as well as the β/γ subunits is induced. G-protein signalling following BLT1/2 activation can then lead to phospholipase C as well as PI₃ kinase activation which can be directly or indirectly responsible for the induction of chemotaxis or enzyme release. LTB₄ can also activate other intracellular kinases of the MAP kinase family such as MEK 1/2, ERK 1/2, p38, as well as JNK. LTB₄ stimulation of human neutrophils can also lead to the activation of the Src family kinase member Yes. Recently, it was also observed that LTB₄ stimulation of human leukocytes leads to the activation of TAK-1 protein.

TLR-, RLR-, as well as NLR-dependent signalling is primarily involved in immune defence mechanisms, but can also be involved in other mechanisms such as tumour suppression and control of inflammatory processes. Since the use of a single compound as a drug for the treatment of human and animal pathologies often leads to partial, yet not complete eradication of the pathology, and that in many cases resistance against such treatment can develop, there is an urgent need for drug combination development protocols.

### SUMMARY OF THE INVENTION

In a first aspect, there is provided a new treatment with LTB₄ in combination with at least one of TLR, RLR, and NLR ligands.

Accordingly, in one aspect, there is provided an innovative immunological tool which consists in the use of LTB₄ in combination with at least one ligand selected from the group consisting of Toll-like receptor ligand, RIG-I-like receptor ligand, and a NOD-like receptor ligand to enhance the immune response.

In another aspect, there is provided the use of a pharmacologically acceptable effective amount of leukotriene B4 (LTB₄) in combination with at least one modulator of a receptor selected from the group consisting of a Toll-like receptor (TLR), a RIG-I-like receptor (RLR), and a NOD-like receptor (NLR), signalling to potentiate an immune response. Alternatively, the modulator of the receptor is a Toll-like receptor (TLR), a RIG-I-like receptor (RLR) or a NOD-like receptor (NLR).

In one aspect, the TLR is TLR 1, TLR2, TLR3, TLR4, TLR5, TLR6, TLR7, TLR8, TLR9, TLR10, a TLR1/2 complex or a TLR2/6 complex. In another aspect the TLR is selected from the group consisting of TLR 1, TLR2, TLR4, TLR5, TLR6. In still another aspect the TLR is TLR3.

In a further aspect, there is provided the use of a pharmacologically acceptable effective amount of leukotriene B4 (LTB₄) with a Toll-like receptor ligand for enhancing an immune response.

The TLR ligand is preferably any one of i) a TLR2 ligand such as for example, without limitation, lipoteichoic acid (LTA), a synthetic tripalmitoylated lipopeptide (PAM₃CSK₄), zymosan, a lipoglycan such as lipoarabinomannan or lipomannan, a peptidoglycan, diacylated lipoprotein MALP-2, synthetic diacylated lipoprotein FSL-1, heat shock protein HSP60, heat shock protein HSP70, heat shock protein HSP96 or high-mobility-group protein 1 (HMG-1), ii) a TLR3 ligand such as for example, without limitation, a double-stranded RNA, a necrotic cell mRNA or a polyinosine-polycytidylic acid (poly I:C), iii) a TLR4 ligand such as for example, without limitation, a lipopolysaccharide (LPS), monophosphoryl lipid A, heat-shock protein HSP22, fibrinogen, fibronectin, a hyaluronan fragment, heparan sulfate, iv) a TLR5 ligand such as for example, without limitation, flagellin, v) a TLR7 or TLR8 ligand, such as for example, without limitation, a single-stranded RNA, an imidazoquinoline such as imiquimod, gardiquimod, and resiquimod, the guanosine analogue loxoribine, a thiazoloquinolone compound or a thymidine homopolymer phosphorothioate oligodeoxynucleotide (Poly(dT)), vi) a TLR9 ligand, such as for example, without limitation, a double-stranded DNA and a cytosine guanine dinucleotide-containing oligodeoxynucleotides (CpG ODN), such as for example without limitation SEQ ID NO:1.

In accordance with another aspect, there is provided the use of a pharmacologically acceptable effective amount of leukotriene B4 (LTB₄), a functional analog or derivative thereof with a RIG-I-like receptor ligand for enhancing an immune response.

In one aspect, the RIG-I-like receptor ligand is preferably selected from the group consisting of retinoic acid-inducible gene-I (RIG-I) ligand, melanoma differentiation-associated gene (Mda5) ligand, and LGP2 ligand. In another aspect, the RIG-I-like receptor ligand is a single-stranded RNA, a double-stranded RNA or a 5′-triphosphate RNA. Alternatively, the RIG-I-like receptor ligand is retinoic acid-inducible gene-I (RIG-I) ligand, melanoma differentiation-associated gene (Mda5) ligand or LGP2 ligand.

In a further aspect, there is provided the use of a pharmacologically acceptable effective amount of leukotriene B4 (LTB4) with a NOD-like receptor ligand for enhancing an immune response. The NOD-like receptor ligand can be without limitation selected from the group consisting of NOD1, NOD2, IPAF, Nalp1b, and Cryopirin/Nalp3 ligand. The NOD-like receptor ligand is preferably meso-diaminopimelic acid, muramyl dipeptide or flagellin. Alternatively, the NOD-like receptor ligand is NOD1, NOD2, IPAF, Nalp1b or Cryopirin/Nalp3 ligand.

In another aspect, the kinase is TAK-1, p38, a Jun N-terminal kinase (JNK kinase) or a combination thereof.

Still in one aspect, there is provided a pharmaceutical composition comprising leukotriene B4 (LTB₄) and at least one modulator of a receptor selected from the group consisting of a Toll-like receptor (TLR), a RIG-I-like receptor (RLR), and a NOD-like receptor (NLR), signalling to potentiate an immune response; or the modulator is a Toll-like receptor (TLR), a RIG-I-like receptor (RLR) or a NOD-like receptor (NLR). The receptors and modulators of same, i.e. their ligands can be those described herein.

Accordingly, there is also provided a method for treating various pathologies as described hereinafter, using the composition described herein.

For the purpose of the present invention the following term is defined below.

The term “LTB₄” is intended to mean to refer to the molecule [5S,12R-6,8,10,14(Z,E,E,Z)-eicosatetraenoic acid] itself or to any functional LTB₄ analogs or functional derivatives thereof. The molecule of LTB₄ and its activity has been well characterized over the year by people skilled in the art, and number of functional variants or analogs have been designed, each of which could be used in combination with ligands of the receptors disclosed herein for obtaining the same results and thus getting the same benefit from the present invention. Thus these functional variants and analogs of LTB₄ are meant to be included in the present invention.

As used herein, a “functional derivative” is a compound which possesses a biological function similar to the compound from wich said derivative is refered to. A molecule is said to be “substantially similar” to another molecule if both molecules have substantially similar structures or if both molecules possess a similar biological activity.

A “fragment” of a molecule is meant to refer to any polypeptide subset of the molecule. Of particular concern to the present invention are fragments which are functional derivatives.

A “variant” of a molecule is meant to refer to a molecule substantially similar in structure and function to the entire molecule. Thus, as the term variant is used herein, two molecules are variants of one another if they possess a similar activity even if the structure of one of the molecules is not found in the other or if the sequence of amino acid residues is not identical.

An “analog” and “ortholog” of a molecule is meant to refer to a molecule substantially similar in function to the entire molecule.

The expression “potentiation of an immune response” shall be given its accepted meaning in the art, i.e. the enhancement of the immune response by increasing the speed and extent of its development and/or by prolonging its duration. Potentiation of an immune response is also intended to mean the accentuation of the response to an immunogen by administration of other substance,

The espression “acceptable effective amount” is meant to refer to the amount of active ingredient necessary to produce a response. Such amount can be readily identified by a person fo the art with simple routine test.

The terms “ligand” and “agonist” are being used herein interchangeably.

### BRIEF DESCRIPTION OF THE SEVERAL VIEWS OF THE DRAWING

FIG. 1 illustrates the effect of LTB₄ on TLR2 mRNA expression by human neutrophils;

FIG. 2 illustrates the effect of LTB₄ on TLR2 protein expression on either untreated human neutrophils (FIG. 2A) or stimulated with LTB₄ (100 nM) for 15 (2), 30 (3), 60 (4) or 120 min (5) (FIG. 2B), LTB₄ alone or in combination with LTA was used;

FIG. 3 illustrates the effect of LTB₄ on TLR7, 8, and 9 mRNA expression by human neutrophils;

FIG. 4 illustrates the effect of LTB₄ on TLR9 protein expression by human neutrophils in a time-dependent manner and compared to other neutrophil ligands, in FIG. 4A, cells were left untreated (dotted line) or were stimulated with LTB₄ (100 nM) for 15, 30, 60 or 120 min; in FIG. 4B, cells were pre-treated for 30 min with the high affinity LTB₄ receptor antagonist U75302 (10 μM) prior to stimulation with LTB₄ for 60 min; in FIGS. 4C-D, cells were stimulated with LTB₄ or leukotrienes C4 (LTC₄), leukotriene D4 (LTD₄), the complement protein C5a or IL-8 for 60 min; in FIG. 4E neutrophils were pretreated with LTB₄ for 60 min and incubated in the presence of CpG-ODN 2216 sequences (SEQ ID NO:1) that were tagged with cyanine-3 (Cy3) fluorochrome; and in FIG. 4F neutrophils were preincubated with LTB₄ for 60 min in the presence of inhibitory CpG-ODN sequences;

FIG. 5 illustrates the enhanced effect of a combination therapy as described herein using LTB₄ and LTA on lipoteichoic acid-mediated TNF-α secretion by human neutrophils using different LTA doses;

FIG. 6 illustrates the enhanced effect of a combination therapy as described herein using LTB₄ and LTA on lipoteichoic acid-mediated TNF-α secretion by human neutrophils using a single LTB₄ dose;

FIG. 7 illustrates the enhanced effect of a combination therapy as described herein using LTB₄ and LTA on lipoteichoic acid-mediated IL-8 secretion by human neutrophils;

FIG. 8 illustrates the enhanced effect of a combination therapy as described herein using LTB₄ and LTA on lipoteichoic acid-mediated TNF-α secretion by human neutrophils in a time-dependent manner;

FIG. 9 illustrates the enhanced effect of a combination therapy as described herein using LTB₄ and LTA on lipoteichoic acid-mediated TNF-α secretion by human neutrophils as dependent on the dose of LTB₄ used;

FIG. 10 illustrates the enhanced effect of a combination therapy as described herein using LTB₄ and PAM₃CSK₄ on PAM₃CSK₄-mediated TNF-α secretion by human neutrophils;

FIG. 11 illustrates the enhanced effect of a combination therapy as described herein using LTB₄ and PAM₃CSK₄ on PAM₃CSK₄-mediated IL-8 secretion by human neutrophils;

FIG. 12 illustrates the enhanced effect of a combination therapy as described herein using LTB₄ and LPS on LPS-mediated TNF-α and IL-8 secretion by human neutrophils, wherein cell-free supernatants were collected and the quantitation of TNF-α (FIG. 12A) or IL-8 (FIG. 12B) was performed using commercially available ELISA assays;

FIG. 13 illustrates the importance of IRAK1- and p38-mediated signaling in the enhanced effect of a combination therapy as described herein using LTB₄ and LTA on LTA-mediated TNF-α secretion by human neutrophils;

FIG. 14 illustrates the enhanced effect of a combination therapy as described herein using LTB₄ with CpG-ODN on CpG-mediated TNF-α secretion by human neutrophils;

FIG. 15 illustrates the enhanced effect of a combination therapy as described herein using LTB₄ with CpG-ODN on CpG-mediated IL-8 secretion by human neutrophils;

FIG. 16 illustrates the enhanced effect of a combination therapy as described herein using LTB₄ with CpG-ODN on CpG-mediated TNF-α secretion by human neutrophils in a time-dependent manner;

FIG. 17 illustrates the enhanced effect of a combination therapy as described herein using LTB₄ on CpG-mediated TNF-α secretion by human neutrophils as dependent on the dose of LTB₄ used;

FIG. 18 illustrates the implication of the high affinity LTB₄ receptor, BLT1, in the enhanced effect of a combination therapy as described herein using LTB₄ with CpG-ODN on CpG-mediated TNF-α secretion by human neutrophils;

FIG. 19 illustrates that simultaneous stimulation with LTB₄ and LTA is a more potent combination therapy than a therapy using LTB₄ in pre-treatment following LTA stimulation on LTA-mediated TNF-α secretion by human neutrophils;

FIG. 20 illustrates the independence of endosomial signaling in the enhanced effect of a combination therapy as described herein using LTB₄ with CpG-ODN on CpG-mediated TNF-α secretion by human neutrophils;

FIG. 21 illustrates the dependence of endosomial signaling in the enhanced effect of a combination therapy as described herein using LTB₄ with CpG-ODN on CpG-mediated TNF-α secretion by peripheral blood mononuclear cells (PMBC);

FIG. 22 illustrates the enhanced effect of a combination therapy as described herein using LTB₄ and LTA on LTA-mediated TAK-1 and p38 activation in human neutrophils in a dose-dependent manner;

FIG. 23 illustrates the enhanced effect of a combination therapy as described herein using LTB₄ and LTA on LTA-mediated IRAK-1 kinase activity in human neutrophils;

FIG. 24 illustrates the enhanced effect of a combination therapy as described herein using LTB₄ and either LTA or Herpes simplex-1 virus (HSV-1) on TNF-α secretion by murine peripheral mononuclear cells, wherein cells were stimulated with a placebo solution (0.45% w/v NaCl containing 0.25% w/v dextrose, NS), LTB₄ (100 nM in placebo solution), LTA (0.1 μg/ml) (FIG. 24A) or the prototypical viral activator or TLR2 herpes simplex virus-1 (HSV-1, 5×10⁴ viral particles) (FIG. 24B) or a combination of either LTB₄ (100 nM in placebo solution) and LTA (0.1 μg/ml) or HSV-1 (5×10⁴ viral particles);

FIG. 25 illustrates the enhanced effect of a combination therapy as described herein using LTB₄ on CpG-mediated TAK-1, p38, and Jun kinase activation by human neutrophils and peripheral blood mononuclear cells, wherein human neutrophils (FIG. 25A) or PBMCs (FIG. 25B) (5×10⁶ cells) were stimulated with a placebo solution (0.45% w/v NaCl containing 0.25% w/v dextrose, NS), LTB₄ (100 nM in placebo solution), CpG-ODN 2216 (10 μg/ml) or a combination of LTB₄ (100 nM in placebo solution) and CpG-ODN 2216 (10 μg/ml);

FIG. 26 illustrates the independence of endosomial signaling in the enhanced effect of a combination therapy as described herein using LTB₄ on CpG-mediated TAK-1, p38, and Jun kinase activation by human neutrophils and peripheral blood mononuclear cells, wherein a combination therapy using LTB₄ in the presence of CpG-ODN enhances intracellular signalling implicating TAK-1, p38, and JNK kinases in both human neutrophils (FIG. 26A) and PBMCs (FIG. 26B) via an endosome-independent pathway; and

FIG. 27 illustrates the enhanced effect of a combination therapy as described herein using LTB₄ on CpG-mediated in vivo antiviral activity directed against murine cytomegalovirus in salivary glands of infected Balb/c mice.

FIG. 28 illustrates the enhanced effect of LTB₄-mediated, RIG-I-dependent NFκB promoter activity in HEK-293T cells stably expressing BLT1 Cells were transiently transfected with a combination of RIG-I-expressing vector and luciferase reporter gene under NFκB promoter control. Promoter activity was recorded following a 24 h stimulation with LTB₄ (1 μM).

FIG. 29 illustrates the enhanced effect of LTB₄-mediated, RIG-I-dependent IFN-β promoter activity in HEK-293T cells stably expressing BLT1. Cells were transiently transfected with a combination of RIG-I-expressing vector and luciferase reporter gene under IFN-β promoter control. Promoter activity was recorded following a 24 h stimulation with LTB₄ (1 μM).

FIG. 30 illustrates the enhanced effect of LTB₄-mediated, IPS-I-dependent NFκB promoter activity in HEK-293T cells stably expressing BLT1. Cells were transiently transfected with a combination of IPS-I-expressing vector and luciferase reporter gene under NFκB promoter control. Promoter activity was recorded following a 24 h stimulation with LTB₄ (1 μM).

FIG. 31 illustrates the enhanced effect of LTB₄-mediated, IPS-I-dependent IFN-β promoter activity in HEK-293T cells stably expressing BLT1. Cells were transiently transfected with a combination of IPS-I-expressing vector and luciferase reporter gene under IFN-β promoter control. Promoter activity was recorded following a 24 h stimulation with LTB₄ (1 μM).

FIG. 32 illustrates that the combined treatment with LTB₄ and the RLR agonist poly I:C potentiates synthesis of IFNβ mRNA levels in A549 cells.

FIG. 33 illustrates that the combined treatment with LTB₄ and the RLR agonist poly I:C potentiates secretion of IFNβ by A549 cells.

FIG. 34 illustrates that combined treatment with LTB₄ and the RLR agonists 3P-RNA or Sendai virus increases synthesis of IFNβ mRNA levels in A549 cells.

FIG. 35 illustrates that combined treatment with LTB₄ and 3P-RNA or Sendai virus potentiates secretion of IFNβ in A549 cells.

FIG. 36 illustrates that treatment with LTB₄ in combination with Sendai virus enhances release of IFNβ by wild type MEFs.

FIG. 37 illustrates that the combination therapy as described herein using LTB₄ and the NLR agonist N-acetyl MDP (NacMDP) synergistically enhances IL-6 secretion by mouse embryogenic fibroblasts (MEF).

FIG. 38 illustrates that the combination therapy as described herein using LTB₄ and the NLR agonist NacMDP enhances IL-6 secretion by IAV-infected MEF.

FIG. 39 illustrates that the combination therapy as described herein using LTB₄ and the NLR agonist NacMDP causes an improved reduction in IAV viral load in infected MEF.

FIG. 40 illustrates that the combination therapy as described herein using LTB₄ and the NLR agonist NacMDP causes an improved reduction in IAV viral load in the lungs of infected mice.

FIG. 41 illustrates that the combination therapy as described herein using LTB₄ and the NLR agonist NacMDP reduces inflammation, as measured by IL-6 secretion, in the lungs of IAV-infected mice.

FIG. 42 illustrates that the combination therapy as described herein using LTB₄ and the NLR agonist NacMDP restores normal lung architecture in IAV-infected mice. Examples of a bronchiole (B) and alveoli (A) are shown. Arrows identify inflammatory cell infiltration.

### DETAILED DESCRIPTION

It is provided herein a new combination therapy using LTB₄ with at least one of a Toll-like receptor (TLR) ligand, a RIG-I-like receptor (RLR) ligand or a NOD-like receptor (NLR) ligand and a new composition for enhancing an immune response response in immunosuppressed patients or in patients suffering from immunological disorders.

### LTB₄

The leukotriene B4 (LTB₄) agent as described herein refers to LTB₄ [5S,12R-6,8,10,14(Z,E,E,Z)-eicosatetraenoic acid] or a functional variant or analog thereof.

### Toll-Like Receptor Ligand

Toll-like receptor ligand, which are known in the art, refers to activators of TLR1/2, TLR2, TLR2/6, TLR3, TLR4, TLR5, TLR7, TLR8, TLR9, and TLR10 signalling. These include, but are not restricted to, lipoteichoic acid, tripalmitoylated lipopeptide (PAM₃CSK₄), zymosan, peptidoglycan, diacylated lipoprotein MALP-2, diacylated lipoprotein FSL-1, heat-shock proteins 22, 60, 70, and 96, HMG-1, double-stranded RNA, polyinosine-polycytidylic acid (poly I:C), lipopolysaccharide, lipid A, fibronectin, fibrinogen, hyaluronan fragments, flagellin, single-stranded RNA, imiquimod, gardiquimod, and resiquimod, loxoribine, thiazoloquinolone compounds, double-stranded DNA, and cytosine guanine dinucleotide-containing oligodeoxynucleotides (CpG ODN). Toll-like receptor ligand also refers to any modified molecules from the aforementioned TLR ligands that can bind to any one of TLR1-10 and lead to intracellular transduced signalling resulting in biological activities. Toll-like receptor ligand also refers to antibodies raised against any one of TLR1-10 that may induce biological activities following binding to their respective TLR molecule. Data are being presented herein that demonstrate the potential for LTB₄ in modulating mRNA levels of TLR2, 7, 8, and 9 in human neutrophils. Moreover, a stimulation with LTB₄ leads to upregulated TLR2 and TLR9 protein expression by human neutrophils.

### RIG-I-like Receptor Ligand

RIG-I-like receptor (RLR) ligand, which are known in the art, refers to activator of RIG-I, Mda5, as well as LGP2 signalling. These ligands include, but are not restricted to, single-stranded RNA, double-stranded RNA, and 5′-triphosphate RNA. RIG-I-like receptor ligand also refers to any modification introduced in an RNA molecule that can lead to binding and activation of RIG-I, Mda5, and LGP2 leading to RLR-like biological activities.

### NOD-like Receptor Ligand

NOD-like receptor (NLR) ligand, which are known in the art, refers to activator of NOD1, NOD2, IPAF, Nalp1b, and Nalp3 signalling. These ligands include, but are not restricted to, meso-diaminopimelic acid, muramyl dipeptide, and flagellin. NOD-like receptor ligand also refers to any modified molecules from the aforementioned NOD-like receptor ligands that can bind to the different members of the NLR family and lead to intracellular transduced signalling resulting in biological activities.

### Combination Therapy

Combination therapy refers to the use of LTB₄ in combination with a specific TLR, RLR or NLR ligand for an enhanced immune response. In human neutrophils, in vitro stimulation with LTB₄ in combination with the TLR2 ligands lipoteichoic acid or PAM₃CSK₄ for 6 h lead to upregulated secretion of the pro-inflammatory cytokines TNF-α and IL-8 when compared to single stimulation with LTB₄ or lipoteichoic acid or PAM₃CSK₄. This enhanced cytokine secretion by combination stimulation was also found to be time- and dose-dependent. The same effect, although to a more impressive extent, is observed in neutrophils, when cells are stimulated with a combination of CpG-ODN and LTB₄. Combination therapy using LTB₄ with LTA or CpG also lead to an upregulation in the activation of TAK-1 kinase. These results demonstrate a potential for the combination therapy for adequate modulation of the immune system. By modulating the immune response, a combination therapy using LTB₄ with a particular TLR, RLR or NLR ligand could be found useful to enhance an innate immune response.

### Pathology

The pathologies that may be treated by combination therapy consisting of LTB₄ agent and either TLR, RLR or NLR ligand can be divided in three main axes namely: suppressed immunity, inflammatory processes and autoimmune cancer.

Innate immunity refers to immunological defence triggered by the non-specific immune response. Many natural TLR agonists such as nucleic acids have been described as possessing immunomodulatory properties. While being potent activators of the innate immunity, treatments with TLR agonists alone partially restore an adequate immune response. For that reason, it becomes highly desirable to be provided with a component showing greater efficacy, and a combination of LTB₄ with any one of a TLR agonist, a RLR agonist, and a NLR agonist is such a candidate.

Inflammatory processes refers to situations where inflammation is uncontrolled leading to the development of pathologies. As mentioned earlier, TLR5 stimulation with flagellin can control gut inflammation mediated by opportunistic infection as well as unbalanced microbial activities of the gut microflora. Combination therapy combining LTB₄ with flagellin therefore provide a new immunomodulatory strategy for the control of gut inflammation and is of determinant efficacy in the control of inflammatory bowel diseases such as Crohn's disease, ulcerative colitis, and Helicobacter pylori-mediated gastric ulcers. TLR signalling has also been associated with the protection of the nervous system. When appropriately controlled, TLR signalling is crucial for preserving central nervous system structure and function whether infection is present or not. Combination therapy with LTB₄ and such TLR ligands is therefore promising for the development of a neuroprotective drug strategy. TLRs are playing an important role in the inflammatory lung disease including acute respiratory distress syndrome, asthma, and chronic obstructive pulmonary disease (COPD). For example, administration of CpG immunostimulatory ODN can directly inhibit allergic response in the lung. The use of TLR ligands has also been proposed to induce protective immunity that may reduce the risk of developing infective exacerbations of COPD. Combination therapy using LTB₄ and TLR ligands is therefore promising in the treatment of lung diseases.

Cancer is the third area where combination therapy has beneficial effects. TLR signalling has been associated with the control of tumor development and metastasis. For example, the TLR2 agonist MALP-2 induces in vitro tumoricidal activity by macrophages. In a pancreatic cancer mouse model, MALP-2 reduced metastases formation in the lung. The TLR4 agonist ONO-4007 (Ono Pharmaceutical Co., Osaka, Japan) has a remarkable and selective efficacy on Tumor necrosis factor α (TNF-α)-sensitive tumours. Imidazoquinoline compounds have also shown therapeutic potential as agents against bladder cancer, leukemia, as well as benign and malignant skin lesions. TLR9 agonists are also in development for cancer therapy. The combination and synergistic therapies is now being proposed in the use of CpG-ODN in order to reach the full clinical potential of such strategies. Therefore, a therapy using LTB₄ and TLR ligands is now being proposed for enhanced cancer regression.

In the present invention, the combination drug therapy described herein utilizes LTB₄ with at least one of a Toll-like receptor (TLR) ligand, a RIG-I-like receptor (RLR) ligand or a NOD-like receptor (NLR) ligand.

In one aspect, the composition of LTB₄ with one of a Toll-like receptor (TLR) ligand, a RIG-I-like receptor (RLR) ligand or a NOD-like receptor (NLR) ligand can be made under different forms such as tablet, capsules, suspension, solution or powder, known to the person of ordinary skilled in the art.

The present invention will be more readily understood by referring to the following examples.

### EXAMPLE I

### Stimulation of Human Neutrophils with LTB₄ Leads to an Upregulation in TLR mRNA and Protein Levels in Neutrophils

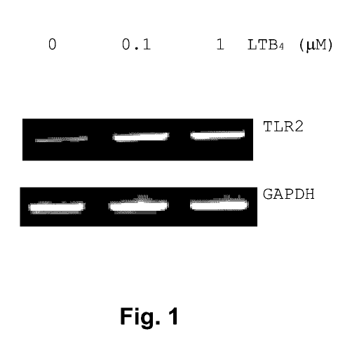

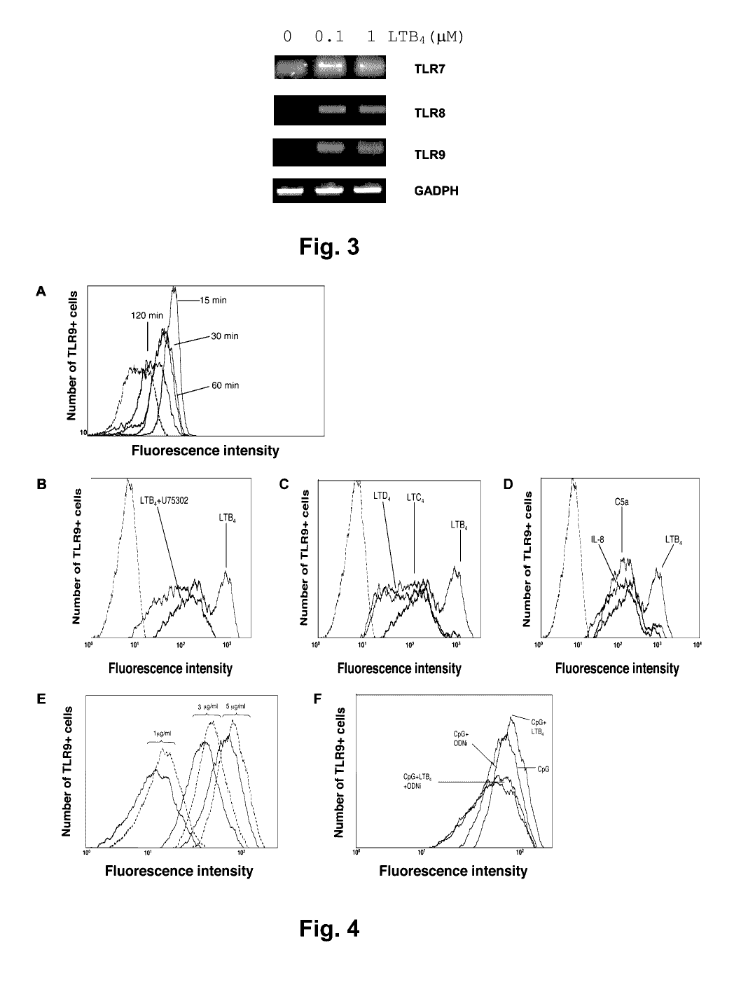

Peripheral blood neutrophils or peripheral blood mononuclear cells were isolated from healthy donors and were purified by centrifugation over Ficoll™ separation medium gradient as already described (Larochelle B, Blood. 1998;92:291-299). Human neutrophils (2×10⁶) were resuspended in culture medium and stimulated with a placebo solution (0.45% w/v NaCl containing 0.25% w/v dextrose, NS) or LTB₄ (100 nM or 1 μM in placebo solution) for two hours. Following stimulation, total RNA was extracted and RT-PCR was performed using specific primers for human TLR2 (FIG. 1) and TLR7-9 (FIG. 3). As can be seen from FIG. 1, expression of TLR2 mRNA is stimulated by LTB₄ in a dose-dependent form (concentration of LTB₄).

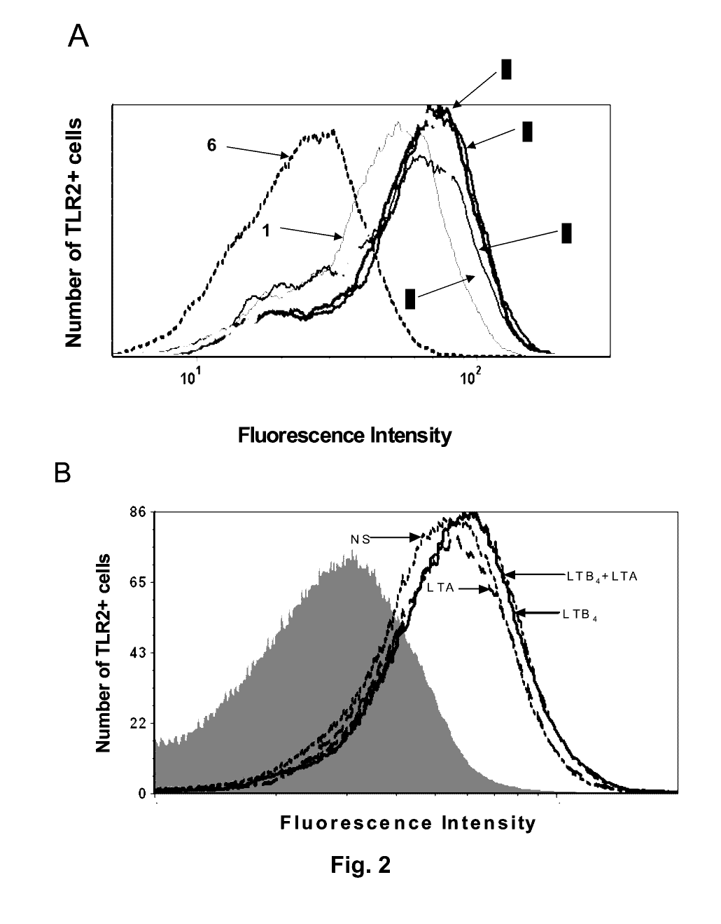

In FIG. 2A, cells were left untreated (1) or were stimulated with LTB₄ (100 nM) for 15 (2), 30 (3), 60 (4) or 120 min (5). Cells were then fixed in paraformaldehyde and TLR2 expression was assessed by flow cytometry. Background cellular autofluorescence is represented by the dotted line (6). In FIG. 2B, LTB₄ alone or in combination with LTA enhanced TLR2 expression. Similarly, in FIG. 3, it can be seen that the mRNA expression of TLR7-9 in cells is stimulated in a dose-dependent form as well by LTB₄.

In FIG. 4A, cells were left untreated (dotted line) or were stimulated with LTB₄ (100 nM) for 15, 30, 60 or 120 min. Background cellular autofluorescence is represented by shaded histogram. Cells were then fixed in paraformaldehyde, permeabilized in methanol, and TLR9 expression was assessed by flow cytometry. In FIG. 4B, cells were pre-treated for 30 min with the high affinity LTB₄ receptor antagonist U75302 (10 μM) prior to stimulation with LTB₄ for 60 min. Cells were then fixed in paraformaldehyde, permeabilized in methanol, and TLR9 expression was assessed by flow cytometry. In FIGS. 4C-D, cells were stimulated with LTB₄ or leukotrienes C4 (LTC₄), leukotriene D4 (LTD₄), the complement protein C5a or IL-8 for 60 min. Cells were then fixed in paraformaldehyde, permeabilized in methanol, and TLR9 expression was assessed by flow cytometry. In FIG. 4E, neutrophils were pretreated with LTB₄ for 60 min. Following this pretreatment, neutrophils were incubated in the presence of CpG-ODN 2216 sequence (5′-G*G*GGGACGATCGTCG*G* G*G*G*G*-3′) (SEQ ID NO:1) that were tagged with cyanine-3 (Cy3) fluorochrome. After an incubation for 4 h with Cy3-CpG-ODN sequences, neutrophils were extensively washed with phosphate buffer and Cy3 expression (corresponding to the degree in CpG-ODN binding on human neutrophils) was assessed by flow cytometry. In FIG. 4F, neutrophils were preincubated with LTB₄ for 60 min in the presence of inhibitory CpG-ODN sequence 5′-TTTAGGGTTAGGGTTAGGGTTAGGG-3′ (phosphothioate as backbone) (SEQ ID NO:3). Following preincubation, Cy3-CpG-ODN binding assay was performed as in FIG. 4E. As can be seen from FIG. 4, LTB₄ induces TLR9 expression on human neutrophils in a time-dependent manner via the high affinity LTB₄ receptor BLT1. LTB₄ is also a potent inducer of TLR9 expression in human neutrophils when compared to other leukotrienes (LTC₄ and LTD₄) or other potent neutrophil agonists such as C5a and IL-8. Moreover, LTB₄ enhances CpG-ODN binding on human neutrophils in a TLR9-dependent manner.

### EXAMPLE II

### Stimulation of Human Neutrophils with a Combination of LTB₄ with TLR2 Ligands Lipoteichoic Acid (LTA) and PAM₃CSK₄ and TLR4 Ligand Lipopolysaccharide (LPS) Leads to an Upregulated Secretion of Pro-inflammatory Cytokines

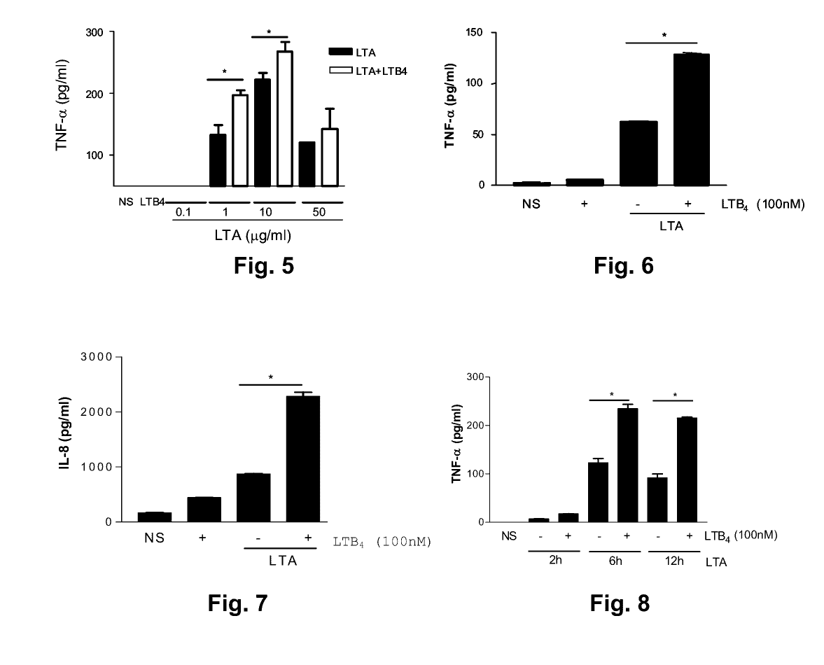

As described in Example I, peripheral blood neutrophils were isolated from healthy donors and were purified by centrifugation over Ficoll™ separation medium gradient. Cells (5×10⁶) were resuspended in culture medium and stimulated with a placebo solution (0.45% w/v NaCl containing 0.25% w/v dextrose, NS), LTB₄ (100 nM in placebo solution), different concentrations of LTA (0.1-50 μg/ml) or a combination of LTA and LTB₄ at their aforementioned respective concentrations. Six hours post-stimulation, cell-free supernatants were collected and the quantitation of TNF-α was performed by ELISA (FIG. 5). As shown in FIGS. 6 and 7, neutrophils were isolated from healthy donors and were purified by centrifugation over Ficoll™ separation medium gradient. Cells (5×10⁶) were resuspended in culture medium and stimulated with a placebo solution (0.45% w/v NaCl containing 0.25% w/v dextrose, NS), LTB₄ (100 nM in placebo solution), LTA at the fixed concentration of 10 μg/ml or a combination of LTA and LTB₄ at their aforementioned respective concentrations. Six hours post-stimulation, cell-free supernatants were collected and the quantification of TNF-α (FIG. 6) or IL-8 (FIG. 7) was performed by ELISA. As can be seen from FIGS. 5-7, LTB₄ can potentiate pro-inflammatory cytokine secretion by human neutrophils stimulated with LTA.

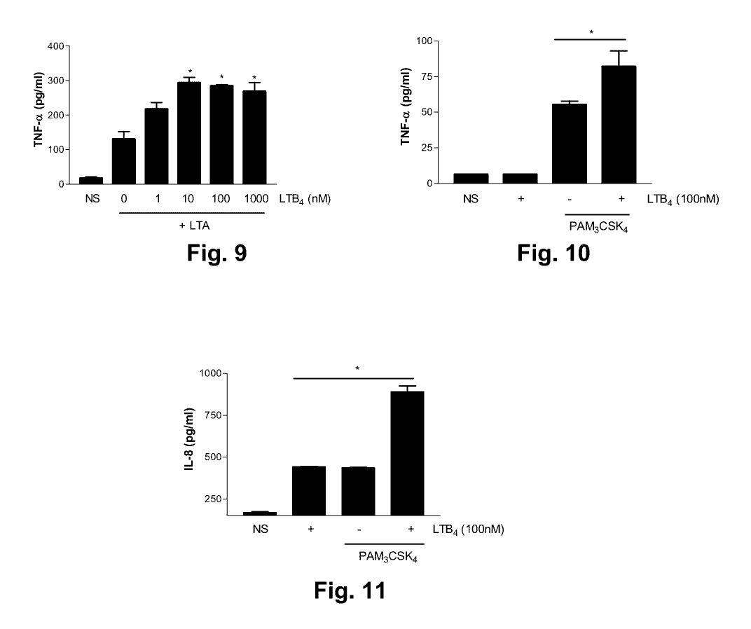

In FIG. 8, cells were prepared as in FIG. 6 and stimulated with LTA for 2 h, 6 h or 12 h before cell-free supernatants were collected for TNF-α detection. In FIG. 9, cells were prepared as in FIG. 6 and were stimulated with LTA in the presence of 0, 1, 10, 100 or 1000 nM of LTB₄. Six hours post-stimulation, cell-free supernatants were collected and the quantification of TNF-α was performed. As can be seen in FIGS. 8-9, LTB₄ can potentiate LTA-mediated TNF-α secretion by human neutrophils in a time- and dose-dependent manner. In FIGS. 10-11, cells (5×10⁶) were resuspended in culture medium and stimulated with a placebo solution (0.45% w/v NaCl containing 0.25% w/v dextrose, NS), LTB₄ (100 nM in placebo solution), PAM₃CSK₄ (10 μg/ml) or a combination of LTB₄ (100 nM in placebo solution) and PAM₃CSK₄ (10 μg/ml). Six hours post-stimulation, cell-free supernatants were collected and the quantification of TNF-α (FIG. 10) or IL-8 (FIG. 11) was performed using commercially available ELISA assays. The symbol “*” represents a probability of p<0.05 when analyzed by one-tailed ANOVA followed by a Newman-Keuls post hoc test using PRISM3 software.

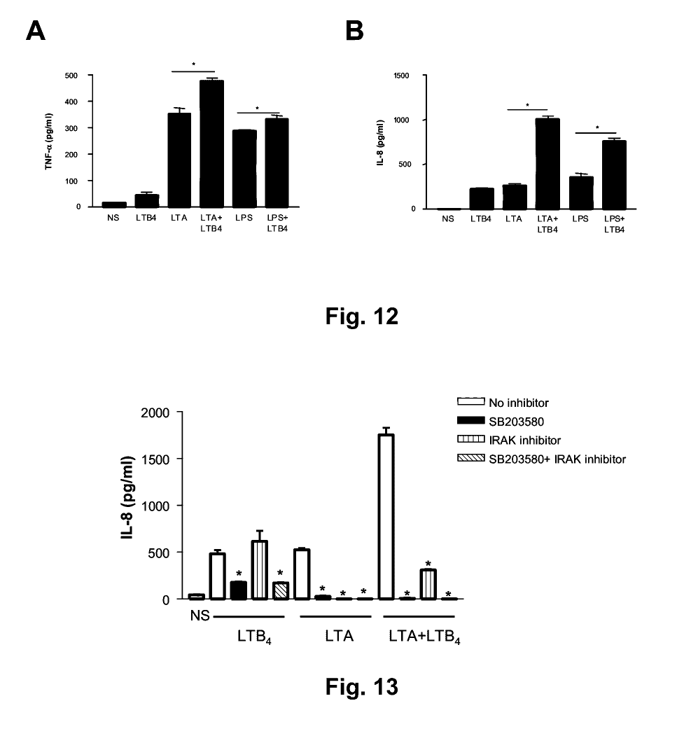

In FIG. 12, neutrophils were prepared as in FIG. 6, and were stimulated with a placebo solution (0.45% w/v NaCl containing 0.25% w/v dextrose, NS), LTB₄ (100 nM in placebo solution), LTA (10 μg/ml), the TLR4 ligand LPS (100 ng/ml) or a combination of LTB₄ (100 nM in placebo solution) and either LTA (10 μg/ml) or LPS (100 ng/ml). Following cell incubation with single ligands or the different ligand combinations for six hours, cell-free supernatants were collected and the quantification of TNF-α (FIG. 12A) or IL-8 (FIG. 12B) was performed using commercially available ELISA assays. The symbol “*” represents a probability of p<0.05 when analyzed by one-tailed ANOVA followed by a Newman-Keuls post hoc test using PRISM3 software. As can be seen in FIGS. 10-12, LTB₄, when administered in combination with different TLR agonists, potentiated TLR-mediated pro-inflammatory cytokine secretion by human neutrophils. In FIG. 13, human neutrophils were pretreated or not with pharmacological inhibitors of p38 kinase (SB203580, 10 μM). IRAK 1/2 kinase inhibitor (IRAK inhibitor, 10 μM) for one hour prior to stimulation with a placebo solution (0.45% w/v NaCl containing 0.25% w/v dextrose, NS), LTB₄ (100 nM in placebo solution), LTA (10 μg/ml) or a combination of LTB₄ in the presence of LTA for 6 hours. Following the incubation with indicated single or combined agonists, cell-free supernatants were collected and the quantification of IL-8 was performed using commercially available ELISA assay. FIG. 13 shows the implication of IRAK1 as well as p38-mediated signalling in LTB₄-induced LTA-mediated cytokine secretion by human neutrophils.

### EXAMPLE III

### Stimulation of Human Neutrophils with a Combination of LTB₄ and TLR9 Ligand CpG Oligodeoxynucleotide (ODN) Leads to an Upregulated Secretion of Pro-inflammatory Cytokines

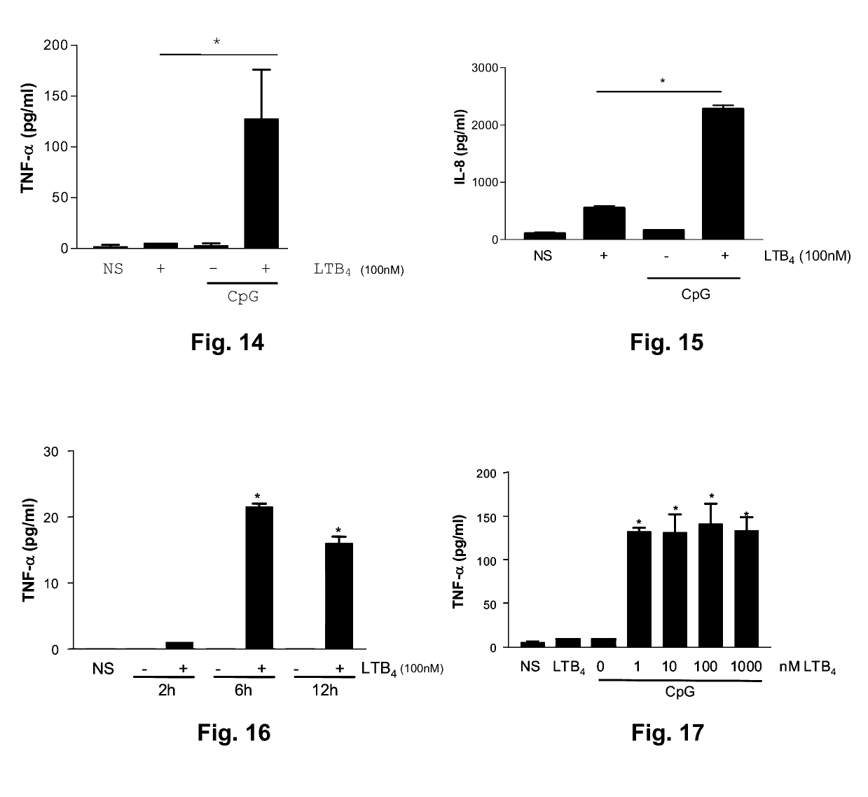

Peripheral blood neutrophils were isolated as already described in Example I. Cells (15×10⁶) were resuspended in culture medium and stimulated with a placebo solution (0.45% w/v NaCl containing 0.25% w/v dextrose, NS), LTB₄ (100 nM in placebo solution), CpG-ODN 2216 (5′-G*G*GGGACGATCGTCG*G*G*G*G*G*-3′) (SEQ ID NO:1), where “*” represents a phosphorothioate linkage) (10 μg/ml) or a combination of LTB₄ (100 nM in placebo solution) and CpG-ODN 2216 (10 μg/ml). Six hours post-stimulation, cell-free supernatants were collected and the quantification of TNF-α (FIG. 14) or IL-8 (FIG. 15) was performed using commercially available ELISA assays. In FIG. 16, cells were prepared as in FIG. 11 and stimulated for 2 h, 6 h or 12 h before cell-free supernatants were collected for TNF-α detection.

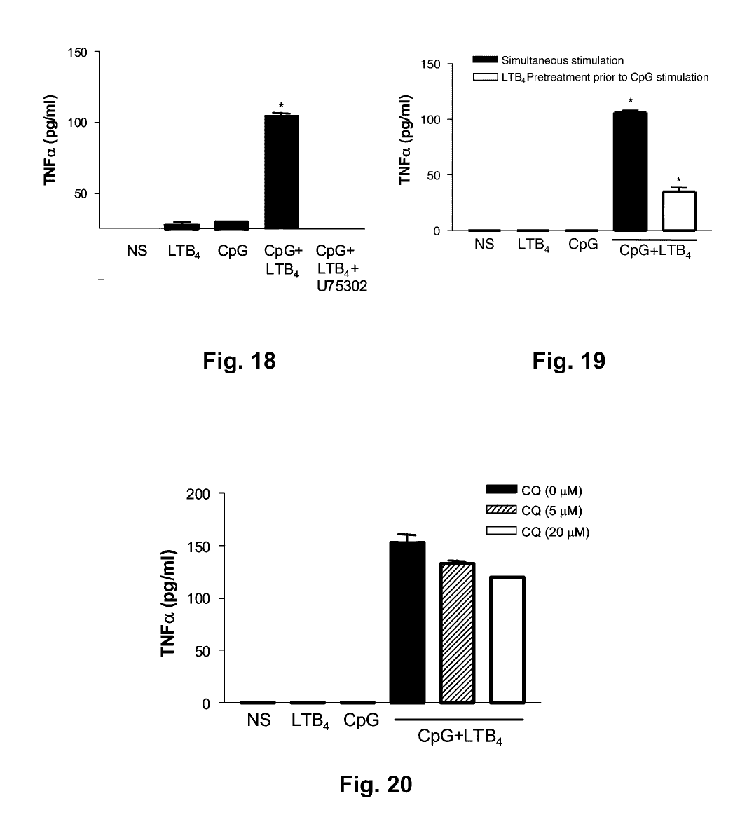

In FIG. 17, cells were prepared as in FIG. 14 and were stimulated with CpG-ODN 2216 (10 μg/ml) in the presence of 0, 1, 10, 100 or 1000 nM of LTB₄. Six hours post-stimulation, cell-free supernatants were collected and the quantification of TNF-α was performed. The symbol “*” represents a probability of p<0.05 analyzed by one-tailed ANOVA followed by a Newman-Keuls post hoc test using PRISM3 software. In FIG. 18, neutrophils were pre-treated for 30 min with the high affinity LTB₄ receptor antagonist U75302 (Cayman, Ann Arbor, Mich.) (10 μM) prior to stimulation with a placebo solution (0.45% w/v NaCl containing 0.25% w/v dextrose, NS), LTB₄ (100 nM in placebo solution), CpG-ODN 2216 (10 μg/ml) or a combination of LTB₄ (100 nM in placebo solution) and CpG-ODN 2216 (10 μg/ml). Six hours post-stimulation, cell-free supernatants were collected and the quantification of TNF-α was assessed by performing a standardized commercially available ELISA assay. In FIG. 19, neutrophils were either pre-treated with LTB₄ prior to CpG-ODN stimulation (10 μg/ml) or cells were simultaneously stimulated with a placebo solution (0.45% w/v NaCl containing 0.25% w/v dextrose, NS), LTB₄ (100 nM in placebo solution), CpG-ODN 2216 (10 μg/ml) or a combination of LTB₄ (100 nM in placebo solution) and CpG-ODN 2216 (10 μg/ml). Six hours post-stimulation, cell-free supernatants were collected and the quantification of TNF-α was assessed by commercial ELISA assay. The symbol “*” represents a probability of p<0.05 analyzed by one-tailed ANOVA followed by a Newman-Keuls post hoc test using PRISM3 software. As seen in FIGS. 14-19, a combined stimulation with LTB₄ and the TLR9 agonist CpG-ODN induces an enhanced time- and concentration-dependent pro-inflammatory cytokine secretion by human neutrophils involving the high affinity LTB₄ receptor BLT1.

### EXAMPLE IV

### LTB₄-mediated Potentiation in CpG Signaling Occurs via Endosomal-dependent Signaling in PBMC, but not in Human Neutrophils

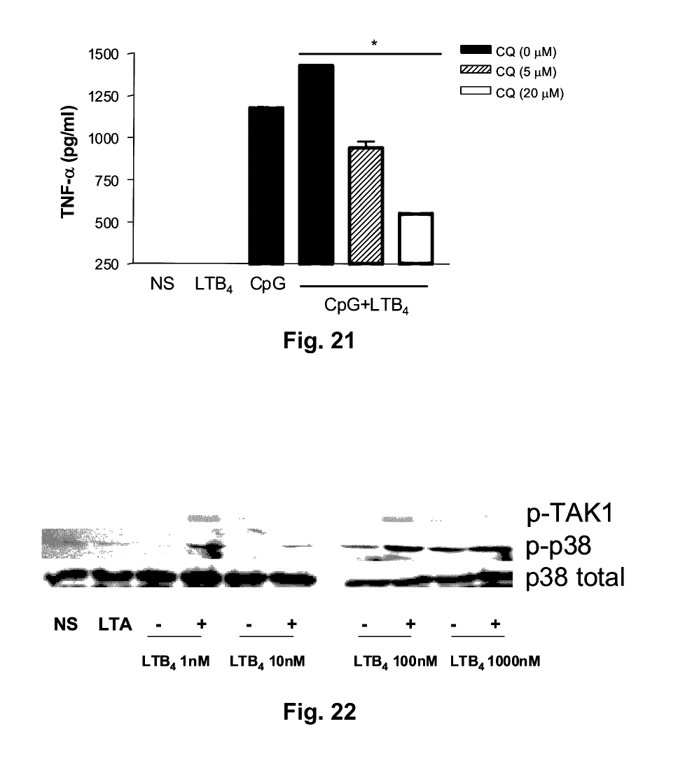

Peripheral blood neutrophils (FIG. 20) or peripheral blood mononuclear cells (PBMC) (FIG. 21) were isolated as already described in Example I. Cells (15×10⁶) were resuspended in culture medium and incubated with chloroquine (CQ), an inhibitor of endosomal activation, for 30 minutes. Following inhibitor incubation, cells were extensively washed with phosphate buffer and were stimulated with a placebo solution (0.45% w/v NaCl containing 0.25% w/v dextrose, NS), LTB₄ (100 nM in placebo solution), CpG-ODN 2216 (10 μg/ml) or a combination of LTB₄ (100 nM in placebo solution) and CpG-ODN 2216 (10 μg/ml). After stimulation for six hours, cell-free supernatants were collected and the quantification of TNF-α was assessed by commercial ELISA assay. The symbol “*” represents a probability of p<0.05 analyzed by one-tailed ANOVA followed by a Newman-Keuls post hoc test using PRISM3 software. As seen in FIGS. 20-21, the combination therapy using LTB₄ and CpG-ODN implicates endosomal signalling in PBMCs (FIG. 21), but not in human neutrophils (FIG. 20).

### EXAMPLE V

### Stimulation of Human Neutrophils with a Combination of LTB₄ and TLR Ligands Leads to an Upregulation in Intracellular Kinase Activation

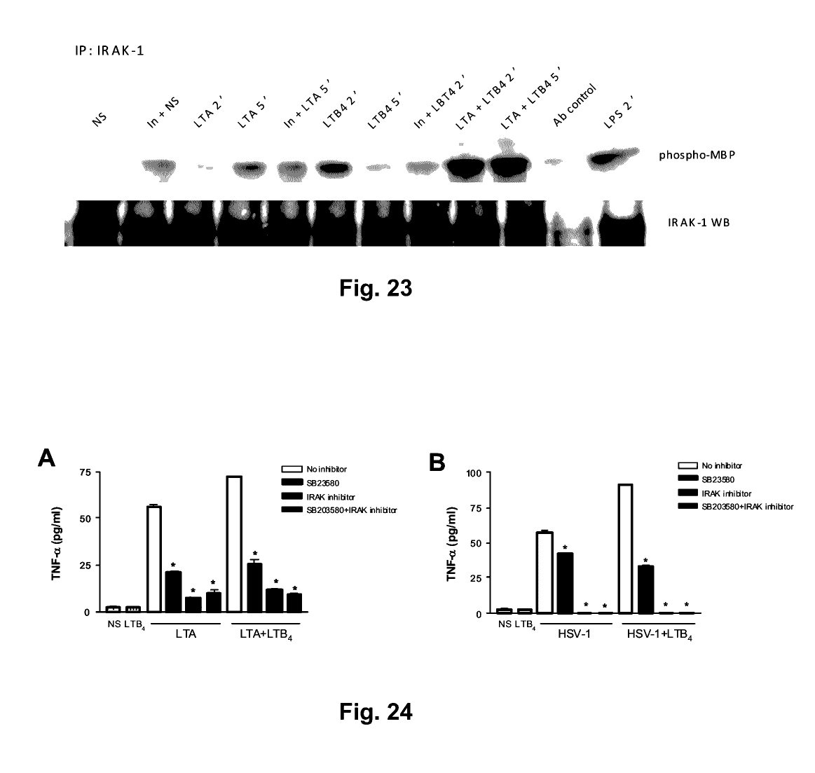

Peripheral blood neutrophils or PBMCs were isolated as already described in Example I. Human neutrophils (5×10⁶) were resuspended in culture medium and stimulated with a placebo solution (0.45% w/v NaCl containing 0.25% w/v dextrose, NS), increasing concentrations of LTB₄ (in placebo solution), LTA (10 μg/ml) or a combination of either LTB₄ and LTA (10 μg/ml) for 30 minutes (FIG. 22). Cell lysates were subjected to Western blotting and levels of phosphorylated TAK1 and p38 were determined. Total p38 levels were also determined as a loading control. In FIG. 23, in vitro IRAK-1 kinase assay was performed. In short, human neutrophils (5×10⁷ cells/ml) were incubated or not with a specific IRAK-1/4 inhibitor (In) for 30 minutes. Following the inhibitor incubation, cells were stimulated with a placebo solution (0.45% w/v NaCl containing 0.25% w/v dextrose, NS), LTB₄ (100 nM in placebo solution), LTA (10 μg/ml) or a combination of either LTB₄ (100 nM in placebo solution) and LTA (10 μg/ml) or with LPS (100 ng/ml) as a positive control.

After 2 or 5 minutes (indicated 2′ and 5′ on the figure), cells were lysed in cold lysis buffer (20 mM Tris pH 7.5, 1% Igepal®, 1 mM EDTA, 150 mM NaCl, 10 mM β-glycerophosphate and protease inhibitor cocktail) by an incubation for 30 minutes on ice. Following lysis, cell lysates were pre-cleared overnight with protein A/G agarose beads and 1 μg of rabbit anti-rat IgG. Free lysates were immunoprecipitated with IRAK-1 antibody or unspecific rabbit IgG and protein A/G agarose beads for 2 h on ice. Beads were washed three times with lysis buffer and three additional times with a kinase assay buffer (20 mM HEPES pH 7.5, 20 mM MgCl2, 3 mM MnCl₂, and 10 mM β-glycerophosphate). A portion of the beads were kept for Western blotting analysis of the quantity of immunoprecipitate IRAK-1 proteins. The remaining of the beads was submitted to IRAK-1 in vitro kinase assay. Beads were incubated for 30 min at 37° C. in kinase assay buffer (50 μl) containing 4 μg of myelin basic protein, a phosphorylation substrate for IRAK-1, 4 μCi of γ-³²P, and 5 μM of ATP for 30 minutes. The reaction was stopped by the addition of 20 μl of sample loading buffer (5× of 50 mM Tris-HCl pH 6.8, 10% glycerol, 2% SDS, 2.5% 13-mercaptoethanol, 0.05% bromophenol blue). Samples were boiled and ran on SDS-PAGE western blot gels.

Phosphorylated MBP (myelin basic protein) bands were detected using X-ray Kodak films or by image analyzer. Total immunoprecipitated IRAK-1 protein was detected by Western blot (WB). FIGS. 22-23 illustrate that a combination therapy using LTB₄ with LTA enhances intracellular signaling pathways implicating IRAK-1, TAK-1, as well as p38 kinases in human neutrophils. In FIG. 24, murine splenocytes were isolated from Balb/c mouse spleens (n=5). Cells (5×10⁶ cells) were incubated with p38 kinase (SB203580) and IRAK-1/4 kinase inhibitors (IRAK-1/4 kinase inhibitor) for 60 minutes. Following incubation with inhibitors, cells were stimulated with a placebo solution (0.45% w/v NaCl containing 0.25% w/v dextrose, NS), LTB₄ (100 nM in placebo solution), LTA (0.1 μg/ml) (FIG. 24A) or the prototypical viral activator or TLR2 herpes simplex virus-1 (HSV-1, 5×10⁴ viral particles) (FIG. 24B) or a combination of either LTB₄ (100 nM in placebo solution) and LTA (0.1 μg/ml) or HSV-1 (5×10⁴ viral particles) and LTB₄ for 24 h. Cell-free supernatants were harvested and murine TNF-α concentrations were evaluated using commercially available ELISA assay. The symbol “*” represents a probability of p<0.05 analyzed by one-tailed ANOVA followed by a Newman-Keuls post hoc test using PRISM3 software. As seen in FIGS. 24A and 24B, a combination therapy with LTB₄ and TLR2 agonists enhances TNF-α release by murine cells.

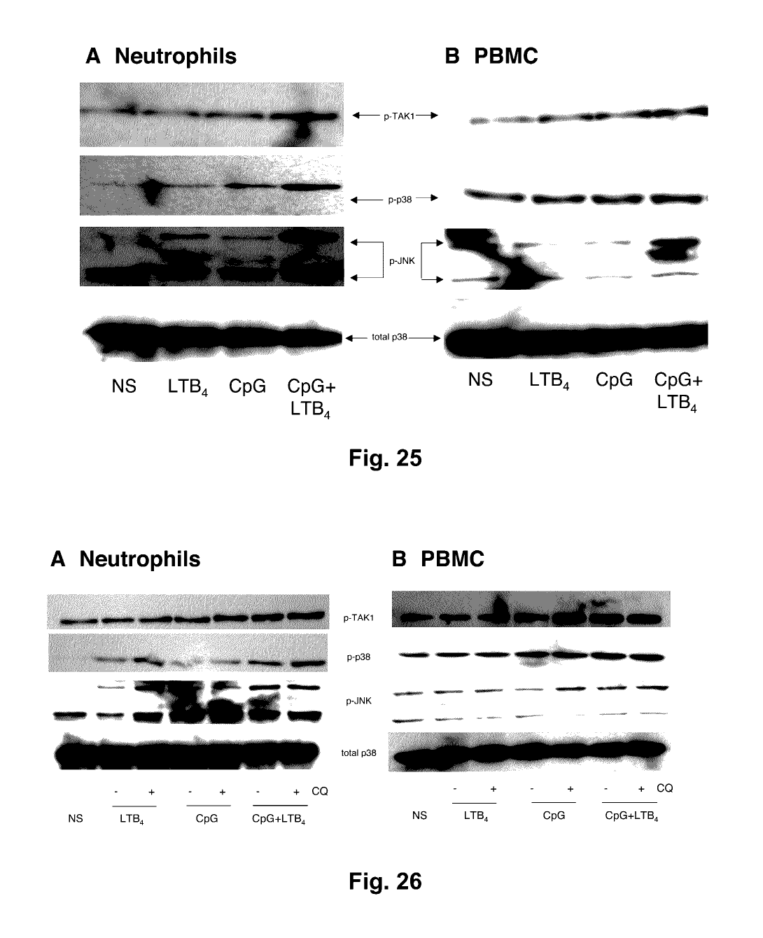

In FIG. 25, human neutrophils (FIG. 25A) or PBMCs (FIG. 25B) (5×10⁶ cells) were stimulated with a placebo solution (0.45% w/v NaCl containing 0.25% w/v dextrose, NS), LTB₄ (100 nM in placebo solution), CpG-ODN 2216 (10 μg/ml) or a combination of LTB₄ (100 nM in placebo solution) and CpG-ODN 2216 (10 μg/ml) for 30 minutes. Following stimulation, cells were lysed and proteins were subjected to Western blotting for determination of phosphorylation levels for TAK1, p38, and Jun kinase (JNK). Total p38 levels were also determined as a loading control. In FIG. 26, cells were prepared as in FIG. 25 and incubated with chloroquine (20 μM) for 30 minutes. Following inhibitor incubation, cells were subjected to a stimulation with a placebo solution (0.45% w/v NaCl containing 0.25% w/v dextrose, NS), LTB₄ (100 nM in placebo solution), CpG-ODN 2216 (10 μg/ml) or a combination of LTB₄ (100 nM in placebo solution) and CpG-ODN 2216 (10 μg/ml) for 30 minutes. Following stimulation, cells were lysed and proteins were subjected to Western blotting for determination of phosphorylation levels for TAK1, p38, and Jun kinase (JNK). Total p38 levels were also determined as a loading control.

As seen in FIGS. 25-26, a combination therapy using LTB₄ in the presence of CpG-ODN enhances intracellular signaling implicating TAK-1, p38, and JNK kinases in both human neutrophils and PBMCs via an endosome-independent pathway.

### EXAMPLE VI

### Administration of a Combination Therapy Including LTB₄ with CpG-ODN Enhances Antiviral Defense in Mice Infected with Murine Cytomegalovirus

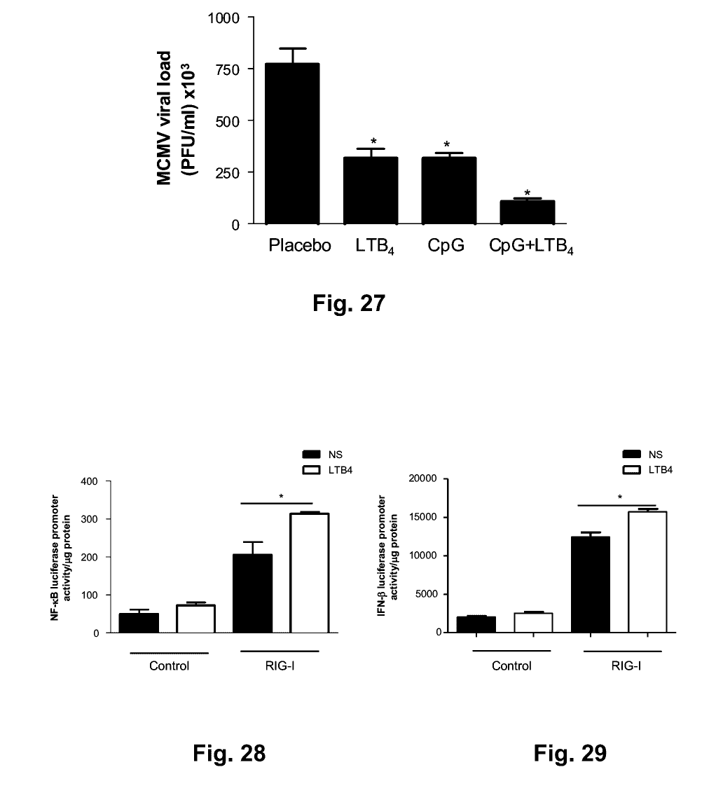

In FIG. 27, four-five week-old female Balb/c mice (15 mice/group) were infected by intraveinous (i.v.) injection of a placebo solution (0.45% w/v NaCl containing 0.25% w/v dextrose) containing murine cytomegalovirus (mCMV) (400 plaque-forming units/mouse). On day 3 post-infection, mice were administered i.v. 100 μl of placebo solution (0.45% w/v NaCl containing 0.25% w/v dextrose), LTB₄ (1000 ng/kg in placebo solution), CpG-ODN 1826 (100 μg in placebo solution) (5-TCCATGACGTTCCGACGTT-3′; SEQ ID NO:2) or a combination of LTB₄ (1000 ng/kg in placebo solution) and CpG-ODN 2216 (100 μg in placebo solution). From day 4 to day 7 post-infection, daily doses of LTB₄ were injected i.v. to groups receiving either LTB₄ alone or LTB₄ in combination with CpG-ODN 1826. Mice were sacrificed by isoflurane overdose on day 8 for salivary gland extraction. Salivary gland murine cytomegalovirus titers were evaluated by a standard plaque assay on mouse embryonic fibroblasts as already reported (Gosselin, J. Immunol., 2005, 174(3): 1587-1593). As seen in FIG. 26, a combination therapy including LTB₄ with CpG-ODN 1826 (SEQ ID NO:2) resulted in enhanced control of murine cytomegalovirus replication when compared to mice treated with a placebo solution, LTB₄ alone or with CpG-ODN alone.

### EXAMPLE VII

### Combination Therapy Including LTB4 and RLR-expressing Vectors or RLR Agonists Enhances RLR-mediated Signaling

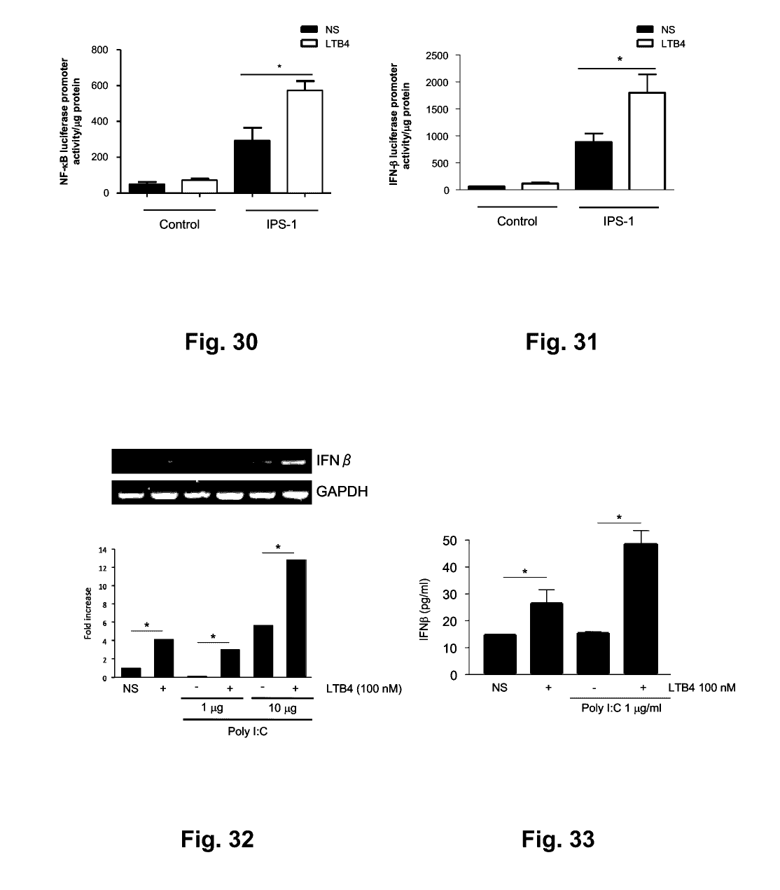

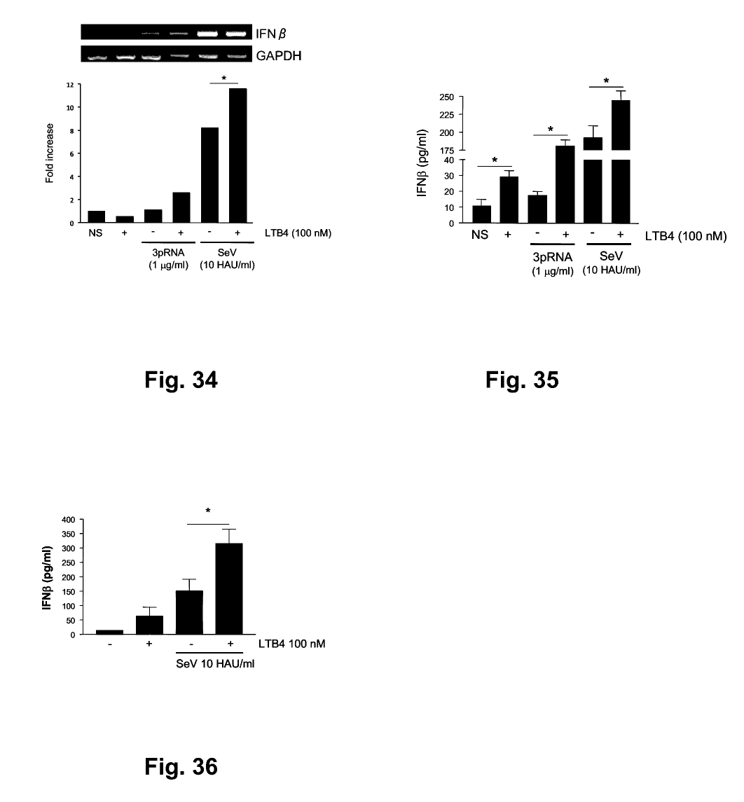

In FIG. 28-31, HEK-293T cell line transfected with BLT1 cDNA was used as a cellular model for promoter activation assays. In FIG. 28-29, BLT1-expressing HEK-293T cells were first co-transfected with control vector or RIG-I-expressing vector (100 ng) along with a vector containing a luciferase gene reporter under NF-κB promoter control (100 ng) (FIG. 28) or IFN-β promoter control (100 ng) (FIG. 29) using Escort V transfection reagent. Briefly, cells were seeded at 5×10⁴ cells per well in a 24-well plate one day prior to transfection. Twenty-four hours post-transfection, cells were stimulated with a placebo solution (0.45% w/v NaCl containing 0.25% w/v dextrose, NS) or LTB₄ (100 nM in placebo solution) for 24 h. Following stimulation, cells were lysed in luciferase buffer (1% triton, 10% glycerol, 20 mM tris phosphate, pH 7.8). Luciferase activity was measured by luminometry and relative light units (RLU) were normalized by protein dosage using BCA protein assay kit. The symbol “*” represents a probability of p<0.05 analyzed by one-tailed ANOVA followed by a Newman-Keuls post hoc test using PRISM3 software. As can be seen in FIG. 28-29, the expression of an active RIG-I form in combination with LTB₄ stimulation enhances NF-κB as well as IFN-β promoter activity. In FIG. 30-31, BLT1-expressing HEK-293T cells were first co-transfected with control vector or IPS-1-expressing vector (100 ng) along with a vector containing a luciferase gene reporter under NF-κB promoter control (100 ng) (FIG. 30) or IFN-β promoter control (100 ng) (FIG. 31) using Escort V transfection reagent. It has to be noted that IPS-1 is a key accessory protein involved in RLR-mediated signaling. Briefly, cells were seeded at 5×10⁴ cells per well in a 24-well plate one day prior to transfection. Twenty-four hours post-transfection, cells were stimulated with a placebo solution (0.45% w/v NaCl containing 0.25% w/v dextrose, NS) or LTB₄ (100 nM in placebo solution) for 24 h. Following stimulation, cells were lysed in luciferase buffer (1% triton, 10% glycerol, 20 mM tris phosphate, pH 7.8). Luciferase activity was measured by luminometry and relative light units (RLU) were normalized by protein dosage using BCA protein assay kit. The symbol “*” represents a probability of p<0.05 analyzed by one-tailed ANOVA followed by a Newman-Keuls post hoc test using PRISM3 software. FIG. 30-31 show that the presence of an active IPS-1 protein in BLT1-expressing cells stimulated with LTB₄ induces NF-κB as well as IFN-β promoter activity, key elements in RLR-mediated signaling. In FIG. 32, lung carcinoma A549 cells (ATCC #CCL-185) were seeded at 1×10⁵ cells per well in a 24-well plate and grown in Ham culture medium supplemented with 10% heat-inactivated fetal bovine serum. Cells were stimulated with synthetic dsRNA poly I:C (1 μg/ml and 10 μg/ml) or with LTB₄ (100 nM) alone or with a combination of synthetic RNA with LTB₄ for 5 hours. Poly I:C was partially digested with dsRNA-specific endonuclease RNase III prior its use. Non stimulated cells (NS) referred to as cells stimulated with a placebo solution (0.45% W/V NaCl containing 0.25% W/V dextrose). The symbol “*” represents a probability of p<0.05 analyzed by one-tailed ANOVA followed by a Newman-Keuls post hoc test using PRISM3 software. After 5 hours of stimulation, total RNA was extracted from samples using TRIZol reagents (Invitrogen) following the manufacturer's instructions. DNase-treated RNA (1 μg) was reverse transcribed using SuperScript reverse transcriptase (Invitrogen) and IFNβ PCR was performed using the specific primers: 5′-GAACTTTGAC ATCCCTGAGG AGATTAAGCA GC-3′ (SEQ ID NO:4) and 5′-GTTCCTTAGG ATTTCCACTC TGACTATGGT CC-3′ (SEQ ID NO:5). GAPDH specific primers 5′-CCACCCATGG CAAATTCCAT GGCA-3′ (SEQ ID NO:6) and 5′-TCTAGACGGC AGGTCAGGTC CACC-3′ (SEQ ID NO:7) were used as internal control. The results presented herein clearly show when cells were stimulated with a combination of RLR agonist (namely the synthetic dsRNA poly I:C) and LTB₄, that mRNA levels of IFNβ were found to be significantly increased (FIG. 32), indicating that LTB₄ potentiates the effects of poly I:C on RLR signaling pathway. In 18 hours stimulated cell samples, cell-free supernatants were harvested and assayed for IFNβ determinations by ELISA (PBL Interferon Source, NJ, USA). As showed in FIG. 33, stimulation with LTB₄ led to an increase in IFNβ levels produced by poly I:C-treated cells. Similarly, mRNA levels of IFNβ as well as secretion of IFNβ by A549 cells was also found to be increased when cells were treated with a combination of LTB₄ and 3P-RNA (1 μg/ml) or with a combination of LTB4 and Sendai virus (FIG. 34-35). These results clearly indicate that LTB₄ potentiates type 1 IFN secretion following stimulation with RLR ligands. Finally, using mouse embryonic fibroblasts (MEFs) isolated from wild type mice and stimulated with LTB₄ alone or in combination with Sendai virus, it is observed that LTB₄ induces secretion of IFNβ but also potentiates secretion of IFNβ in MEFs stimulated with Sendai virus (FIG. 36), indicating that LTB₄ can exert its action through RLR signaling pathway.

### EXAMPLE VIII

### Synergistic Pro-inflammatory Cytokine Secretion by LTB4 and N-acetyl Muramyl Dipeptide in Mouse Embryogenic Fibroblasts

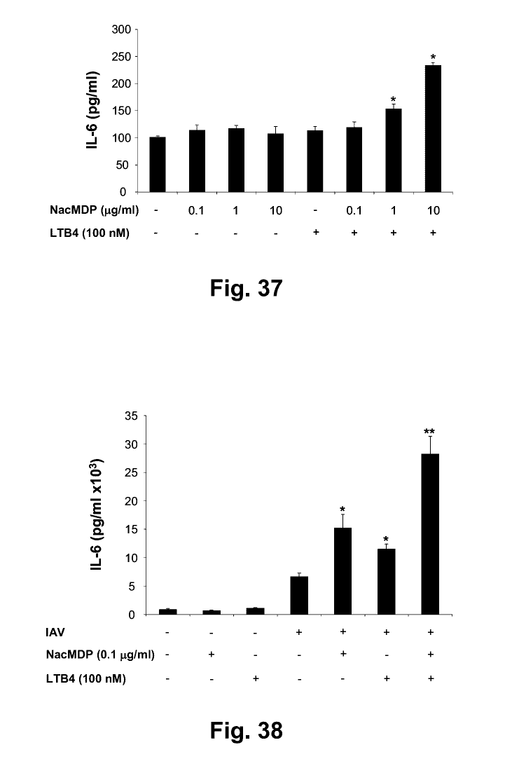

To illustrate the effect of the combination of LTB₄ with a NLR agonist, the NOD2 NLR agonist N-acetyl muramyl dipeptide (NacMDP) was used. For in vitro experiments, mouse embryogenic fibroblasts (MEF) were derived from 14-day C57131/6 mice embryos and expanded in Minimum Essential Medium Eagle (MEM) containing 10% Fetal Bovine Serum (FBS). Cells were then resuspended in culture medium and allowed to adhere overnight in 24-well plates (1×10⁵ cells/well). Following incubation, cells were washed once with HBSS and were either left unstimulated in culture medium or stimulated for 6 hours with increasing concentrations of NacMDP alone (0.1 μg/ml, 1 μg/ml and 10 μg/ml), LTB₄ alone (100 nM) or a combination of LTB₄ and increasing concentrations of NacMDP (FIG. 37). Following stimulation, cell culture supernatants were harvested and assayed for IL-6 determination by ELISA. As can be seen from FIG. 37, NacMDP alone and LTB₄ alone do not induce significant IL-6 secretion compared to background, however, when used in combination, they cause a significant synergistic increase in IL-6 secretion at 1 and 10 μg/ml NacMDP. In FIG. 38, cells were either left uninfected or infected for 16 hours with Influenza A/PR/8/34 H1N1 (IAV) alone (0.1 m.o.i.) or in the presence of either NacMDP alone (0.1 μg/ml), LTB₄ alone (100 nM) or a combination of NacMDP and LTB₄. Following stimulation/infection, cell culture supernatants were harvested and assayed for IL-6 determination by ELISA. In IAV-infected cells (FIG. 38), both NacMDP alone and LTB4 alone significantly enhance IL-6 secretion compared to infected but untreated cells. This synergistic enhancement becomes additive when both compounds are used in combination. In FIG. 37, * p≦0.05 compared to unstimulated cells or cells stimulated with increasing concentrations of NacMDP alone or LTB₄ alone. In FIG. 38, * p≦0.05 compared to cells infected with IAV alone and ** p≦0.05 compared to cells infected with IAV alone or in the presence of NacMDP alone or LTB₄ alone.

### EXAMPLE IX

### Synergistic Antiviral Activity of LTB4 and N-acetyl Muramyl Dipeptide Against Influenza Virus in vitro and in vivo

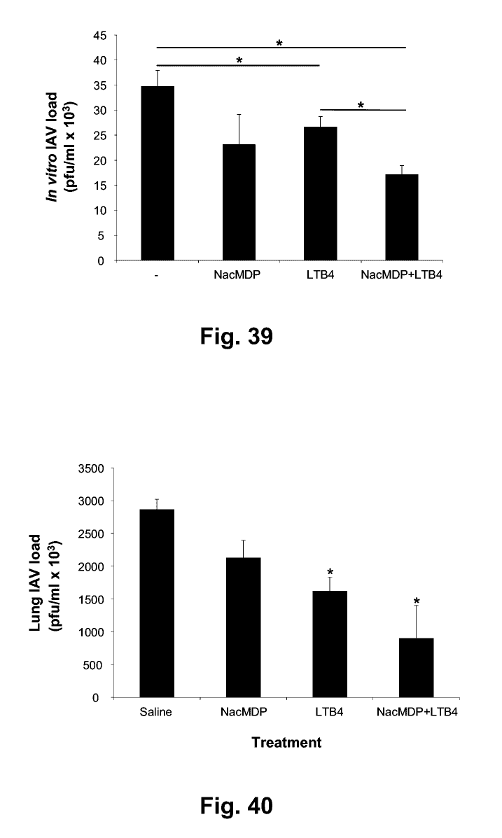

In FIG. 39, MEFs were generated and plated as described in Example VII. Cells were infected with IAV (0.1 m.o.i.) alone (−), LTB₄ alone (100 nM) or with a combination of LTB₄ and NacMDP (0.1 μg/ml) for 24 hours. Following infection, cell culture supernatants were harvested and assayed for viral load determination by standard plaque assay in MDCK cells. For in vivo assessment of the antiviral activity of LTB₄ and NacMDP, 4-6 weeks old C57131/6 female mice (n=4/group) were infected intranasally (in.) with 50 plaque-forming units (pfu) of IAV (FIG. 40). Starting at day one post-infection, mice were treated daily for three days with either saline, LTB₄ alone (1 μg/kg) or with a combination of LTB₄ and NacMDP (2 mg/kg), via the intravenous (iv.) route. Four days post-infection, animals were sacrificed and viral loads were determined from triturated lungs by standard plaque assay in MDCK cells. Both in vitro (FIG. 39) and in vivo (FIG. 40), LTB₄ treatment causes a significant decrease in viral load, which is synergistically enhanced upon combination with NacMDP. In FIG. 39, * p≦0.05. In FIG. 40, * p≦0.05 compared IAV-infected mice receiving saline as treatment.

### EXAMPLE X

### Reduced Inflammation in the Lungs of Influenza-infected Mice Treated with a Combination of LTB4 and N-acetyl MOP

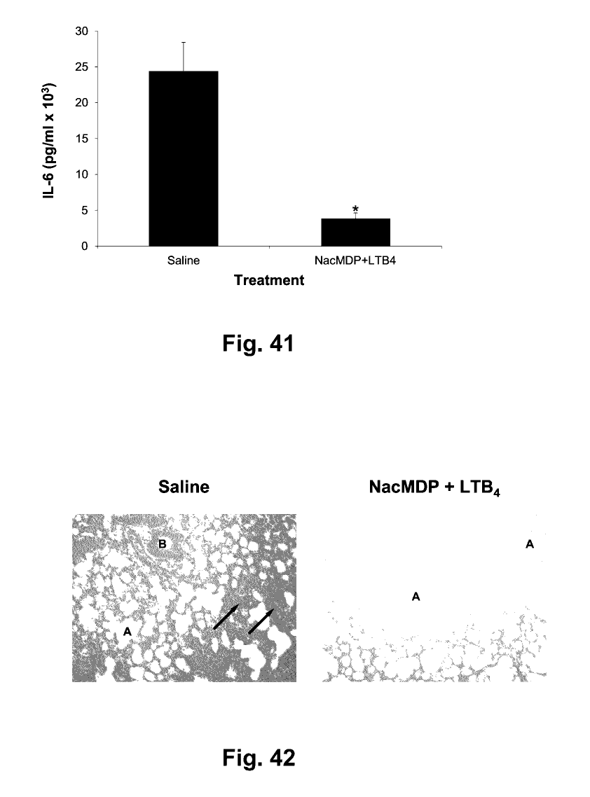

To further investigate the in vivo consequences associated with the treatment of IAV-infected mice with a combination of LTB₄ and the NOD2 agonist NacMDP, 4-6 weeks old C57131/6 female mice (n=4/group) were infected as described in Example IX and treated with either saline or a combination of LTB₄ and NacMDP also as described. In FIG. 41, four-day infected animals were sacrificed and IL-6 titers were determined from triturated lungs by ELISA. In FIG. 42, hematoxylin & eosin stains were performed on pulmonary lobes from four-day infected mice treated daily with saline or a combination of LTB₄ and NacMDP as described. As can be seen in FIGS. 41 and 42 the antiviral activity of the combined LTB₄/NacMDP treatment shown in Example IX is accompanied by a significant reduction in pro-inflammatory IL-6 secretion and was also found to restore normal lung architecture of IAV-infected mice at day three post-treatment initiation. In FIG. 41, * p≦0.05 compared to IAV-infected mice receiving saline as treatment.

While the invention has been described in connection with specific embodiments thereof, it will be understood that it is capable of further modifications and this application is intended to cover any variations, uses or adaptations of the invention following, in general, the principles of the invention and including such departures from the present disclosure as come within known or customary practice within the art to which the invention pertains and as may be applied to the essential features hereinbefore set forth.

1. A method for triggering an innate immune response in a patient comprising administering to said patient a pharmacologically acceptable effective amount of leukotriene B₄ (LTB₄) in combination with at least one modulator of a receptor selected from the group consisting of a Toll-like receptor (TLR), a RIG-I-like receptor (RLR), and a NOD-like receptor (NLR).

2. The method according to claim 1, wherein said Toll-like receptor is selected from the group consisting of TLR1 to TLR10.

3. The method according to claim 1, wherein said Toll-like receptor is a TLR1/2 complex.

4. The method according to claim 1, wherein said Toll-like receptor is a TLR2/6 complex.

5. The method according to claim 1, wherein said Toll-like receptor is a TLR1, TLR2, TLR4, TLR5 or TLR6.

6. The method according to claim 1, wherein said Toll-like receptor is a TLR3.

7. The method according to claim 1, wherein the modulator of said Toll-like receptor is a TLR2 ligand, lipoteichoic acid (LTA), a synthetic tripalmitoylated lipopeptide (PAM3CSK4), zymosan, a lipoglycan, a lipoarabinomannan, a lipomannan, a peptidoglycan, a diacylated lipoprotein MALP-2, a synthetic diacylated lipoprotein FSL-1, a heat shock protein HSP60, a heat shock protein HSP70, a heat shock protein HSP96, a high-mobility-group protein 1 (HMG-1), a TLR3 ligand, a double-stranded RNA, a necrotic cell mRNA, a polyinosine-polycytidylic acid (poly I:C), a TLR4 ligand, a lipopolysaccharide (LPS), a monophosphoryl lipid A, a heat-shock protein HSP22, a fibrinogen, a fibronectin, a hyaluronan fragment, a heparan sulphate, a TLR5 ligand, flagellin, a TLR7 ligand, a TLR8 ligand, a single-stranded RNA, an imidazoquinoline compound, a guanosine analogue loxoribine, a thiazoloquinolone compound, a thymidine homopolymer phosphorothioate oligodeoxynucleotide (Poly(dT)), a TLR9 ligand, a double-stranded DNA, a cytosine guanine dinucleotide-containing oligodeoxynucleotides (CpG ODN) or a combination thereof.

8. The method according to claim 7, wherein said imidazoquinoline compound is imiquimod, gardiquimod or resiquimod.

9. The method according to claim 7, wherein said CpG ODN is SEQ ID NO:1.

10. The method according to claim 1, wherein the modulator of said RIG-I-like receptor is a retinoic acid-inducible gene-I (RIG-I) ligand, a melanoma differentiation-associated gene (Mda5) ligand, a LGP2 ligand, a single-stranded RNA, a double-stranded RNA, a 5′-triphosphate RNA or a combination thereof.

11. The method according to claim 1, wherein the modulator of said NOD-like receptor is IPAF, Nalp1, Cryopirin/Nalp3 ligand or a combination thereof.

12. The method according to claim 1, wherein the modulator of said NOD-like receptor is meso-diaminopimelic acid, muramyl dipeptide (MDP) or a derivative thereof, flagellin or a combination thereof.

13. A method for stimulating neutrophils in a patient comprising administering to said patient a pharmacologically acceptable effective amount of leukotriene B₄ (LTB₄) in combination with at least one modulator of a receptor selected from the group consisting of a Toll-like receptor (TLR), a RIG-I-like receptor (RLR), and a NOD-like receptor (NLR).

14. A method for stimulating secretion of a pro-inflammatory cytokine in a patient comprising administering to said patient a pharmacologically acceptable effective amount of leukotriene B₄ (LTB₄) in combination with at least one modulator of a receptor selected from the group consisting of a Toll-like receptor (TLR), a RIG-I-like receptor (RLR), and a NOD-like receptor (NLR).

15. A method for stimulating intracellular kinase activation in a patient comprising administering to said patient a pharmacologically acceptable effective amount of leukotriene B₄ (LTB₄) in combination with at least one modulator of a receptor selected from the group consisting of a Toll-like receptor (TLR), a RIG-I-like receptor (RLR), and a NOD-like receptor (NLR).

16. The method of claim 15, wherein said kinase is TAK-1, p38, a c-Jun N-terminal kinase (JNK kinase) or a combination thereof.

17. A method for stimulating release of Tumor necrosis factor a (TNF-α) in a patient comprising administering to said patient a pharmacologically acceptable effective amount of leukotriene B₄ (LTB₄) in combination with at least one modulator of a receptor selected from the group consisting of a Toll-like receptor (TLR), a RIG-I-like receptor (RLR), and a NOD-like receptor (NLR).

18. A method for treating a viral infection in a patient comprising administering to said patient a pharmacologically acceptable effective amount of leukotriene B₄ (LTB₄) in combination with at least one modulator of a receptor selected from the group consisting of a Toll-like receptor (TLR), a RIG-I-like receptor (RLR), and a NOD-like receptor (NLR).

19. The method according to claim 18, wherein said viral infection is from cytomegalovirus.


SEQUENCE LISTING (7 sequences)

SEQ ID NO: 1 - DNA, 20 bp, Artificial Sequence
  CpG-ODN 2216
gggggacgatcgtcgggggg

SEQ ID NO: 2 - DNA, 19 bp, Artificial Sequence
  CpG-ODN 1826
tccatgacgttccgacgtt

SEQ ID NO: 3 - DNA, 25 bp, Artificial Sequence
  inhibitory CpG-ODN
tttagggttagggttagggttaggg

SEQ ID NO: 4 - DNA, 32 bp, Artificial Sequence
  IFN-beta forward PCR primer
gaactttgacatccctgaggagattaagcagc

SEQ ID NO: 5 - DNA, 32 bp, Artificial Sequence
  IFN-beta reverse PCR primer
gttccttaggatttccactctgactatggtcc

SEQ ID NO: 6 - DNA, 24 bp, Artificial Sequence
  GAPDH specific PCR forward primer
ccacccatggcaaattccatggca

SEQ ID NO: 7 - DNA, 24 bp, Artificial Sequence
  GAPDH specific PCR reverse primer
tctagacggcaggtcaggtccacc